(19) United States
(12) Patent Application Publication
(10) Pub. No.: US 2010/0189648 A1
(43) Pub. Date: Jul. 29, 2010

(54) INHIBITORS FOR DISRUPTING THE INTERACTION OF UBIQUITINATION RELATED ENZYMES AND USES THEREOF

(76) Inventors: Lan Huang, Beijing (CN); Li Xing, Beijing (CN)

Correspondence Address: KNOBBE MARTENS OLSON & BEAR LLP, 2040 MAIN STREET, FOURTEENTH FLOOR, IRVINE, CA, 92614, US

(21) Appl. No.: 12/513,154

(22) PCT Filed: Nov. 1, 2007
(86) PCT No.: PCT/CN07/03105

§ 371 Date: Feb. 11, 2010

Related U.S. Application Data
(60) Provisional application No. 60/856,125, filed on Nov. 2, 2006.

(51) Int. Cl. A61K 31/437; C07D 471/04; C07D 231/18; A61K 31/415; C07D 239/93; A61K 31/517; C07D 209/20; A61K 31/404; A61K 49/00; A61P 35/00; G06G 7/48; C12N 9/00

(52) U.S. Cl. 424/9.1; 546/113; 514/300; 548/370.4; 514/407; 544/289; 514/266.31; 548/503; 514/419; 703/11; 435/183

(57) ABSTRACT

A hydrophobic binding pocket on ubiquitin-protein ligase E3 is described, and used in designing the inhibitors disrupting ubiquitin conjugating enzyme E2 and E3 interaction. Four types of inhibitors designed by using the binding pocket are provided, which can be used for cancer treatment.

### FIELD OF THE INVENTION

The present invention relates to structure-based drug design in ubiquitination pathways and using these chemical compounds for treatment of cancer. In particular, the present invention relates to the identification of these classes of inhibitors of E2-E3, a highly regulated protein-protein surface interaction. The compositions of these classes of compounds and their analogs are protected.

The invention is applicable to cancers and other diseases general in mammals. The reference to human biochemistry is not intended to be limiting, but illustrative. The term patient or body or reference to humans is utilized for convenience, but includes all mammalian patients or bodies.

### BACKGROUND OF THE INVENTION

### Introduction

Ubiquitin-mediated degradation is a protein regulatory mechanism that controls protein abundance at a post-translational level. It is as significant a control mechanism as phosphorylation. This pathway plays important roles in DNA repair, cell cycle control, class I antigen processing, chromosomal organization, signal transduction, receptor mediated endocytosis, and apoptosis (Hochstrasser, M. 1996). Mis-regulation of these processes can cause uncontrolled cell growth, or cancer, including colon cancer, breast cancer, lung cancer, leukemia, lymphoma, and myeloma (Konstantinopoulos P A. et al.; 2006starita L M et al; Montagut C. et al.; Pulczynski, S; Keating, M. J.; Hall, E. J.; Magrath, I.; Voutsadakis, I. A.).

The ubiquitination cascade involves the successive action of E1, E2 and E3 activities (Scheffner, M. et al, 1998). The E1 (ubiquitin-activating) enzyme, in an ATP-dependent reaction, activates ubiquitin by forming a thioester bond between its active site cysteine and the carboxyl-terminus of ubiquitin. Ubiquitin is then transferred to the active site cysteine of E2 (ubiquitin-conjugating) enzymes, maintaining a thioester linkage. E3 ubiquitin-protein ligases are minimally defined as additional proteins or protein complexes necessary for the recognition and ubiquitination of specific substrates. E3 mediates the transfer of ubiquitin from the E2 to the lysine side chains, first on the substrate, subsequently on ubiquitin. Finally, the poly-ubiquitinated substrates are recognized by the proteasome for degradation. There are two classes of E3: the RING finger E3 family, which have not been shown to form thioester linkage with ubiquitin (Scheffner, M. et al).; and the Hect domain E3 ligase family (Schwarz, S. E. et al.). Hect domain stands for homologous to E6-associated protein [E6AP]carboxyl-terminal domain. This Hect class of E3 proteins forms ubiquitin thioester intermediates and promotes poly-ubiquitination of the substrates.

Both RING finger E3 and HECT E3 interact with E2 (all E2 displays the same structure) in similar structural manner (Huang, L et al, Zheng, N. et al.) But there are subtle differences in the binding pocket (seen in FIG. 1). Thus inhibitors of this surface could affect the ubiquitination of substrate of both classes of E3 ligases with selectivity. These inhibitors will have many applications in a variety of diseases. It is very difficult to disrupt protein-protein interaction. However, this E2-E3 case is quite unique. According to Inventor's biochemical studies, the binding between E2 (UbCH₇) and E3 (E6AP's Hect domain) was quite weak, not withstanding the gel filtration force. The binding could only be detected in native gel electrophoresis and co-crystallization. Furthermore, conducted by Inventor, thorough analysis of the interface of the E2-E3 complexes revealed a well-defined hydrophobic pocket that appeared critical for the coupling interactions of the E2-E3 complex. According to the size, shape and nature of the hydrophobic pocket the binding is best suited for a small molecule target. Thus the interaction point between E2 and E3 could easily be disrupted by small molecules, which forms a basis for this invention.

One famous substrate for ubiquitination pathways is the “genome guardian” tumor suppressor P53. The inactivation of the tumor suppressor p53 has the highest incidence in cancer. Following exposure to genotoxic agents, activation of the transcription factor p53 prevents replication of damaged DNA by either instituting cell cycle arrest at G1/S checkpoint or by targeting the damaged cells for apoptosis (Oren, M.). Inactivation of p53 occurs in more than 50% of all human cancers. For example, p53 mis-regulation is involved in 35% of Burkitt's lymphoma and 60% of L3 type B-cell acute lymphoblastic leukemia (Soussi, T. & Jonveaux, P.). In addition to p53 mutation, ubiquitin-mediated degradation of p53 is one of the efficient ways to inactivate p53, and it frequently occurs in leukemia and lymphoma (Masdehors, P. et al.; Bueso-Ramos, C. E. et al.; Teoh, G. et al.; Pan, Y. & Haines, D. S.; Gustafsson, B. & Stal, O.). P53 degradation is mediated through both classes of E3 ligases in different cellular context. In normal cells, a RING finger E3 ligase MDM2 regulates p53 level by degradation (Fuchs, S. Y. et al; Honda, R.). In human papillomavirus (HPV) infected cells, E6AP, “hijacked” by viral protein E6, ubiquitinates p53. Thus inhibition of the degradation of tumor suppressor P53 through blocking the E2-E3 binding and thus ubiquitin transfer to P53 could potentially inhibitor tumor growth.

Other diseases, such as diabetes and neuro-degenerative diseases are implicated to be caused by mal-function of ubiquitination pathways. Inappropriate degradation of insulin signaling molecules such as insulin receptor substrates (IRS-1 and IRS-2) has been demonstrated in experimental diabetes (Balasubramanyam M. et al.) A series of recent reports have evaluated the pre-clinical efficacy of the proteasome inhibitor MLN519 for the treatment of focal ischemic/reperfusion brain injury in rats (Williams A J, et al.).

The present invention provides novel chemical compound classes that were discovered using structure-based drug design techniques, based on ubiquitination cascade E2-E3 complex crystal structures. E2 and E3 are important and necessary enzymes for protein degradation. Malfunction of protein degradation cause many serious diseases. Protein ubiquitination is as important as protein phosphorylation. Thus the importance of E2 and E3 enzymes rivals that of kinases, which inhibitors are developed as novel drugs on the market. We also claim our classes of novel compounds and their analogs useful for the treatment of cancer, such as breast cancer, colon cancer, cervical cancer, lung cancer, liver cancer, and brain tumor.

### SUMMARY OF THE INVENTION

One object of this invention is to define the specific pocket of the E2-E3 binding interface that is suitable for binding of a small therapeutic agent, thus effective at disrupting the E2-E3 complex formation leading to the therapeutic effect controlled by the E2-E3 signaling pathway (seen in FIGS. 1 and 2).

Another object of the present invention is to provide processes, which were used to identify compound classes that fit into E3′ s E2 binding pocket.

Another object of the present invention is to provide the compositions of these classes of compounds.

An additional object of the invention is to show these classes of compounds useful in inhibition of cancer cell growth, and thus will be useful in the treatment of subjects suffering from cancer comprising these classes of compounds or analogs thereof alone, or in combination with other chemotherapy agents or pharmaceutical carriers.

Therefore, on one aspect, the present invention provides a protein binding pocket for design E2 inhibitors comprising Val657, Leu658, Ser661, Leu662, Leu665, Met 676, Ile678, Ile682, Ile705, Phe713, Tyr717 based on SEQ ID NO. 1.

On the second aspect, the present invention provides a process for designing E2 inhibitors, wherein the process comprising using the said protein binging.

On the third aspect, the present invention provides a E2 inhibitor, wherein said inhibitor comprises a compound selected from the following groups:

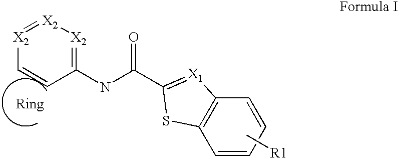

### X1=N, C

R1=halogens (F,Cl,Br), Me, Ome, OH mono- or multi-substituted at the free valences using combinations of the functional groups
X2=one of more of it could be either C or N
Ring=five- or six-membered rings fuzed with the aromatic ring explicitly drawn, including benzene, pyridine, pyrimidine, pyrizine, pyrrole, imidazole, furan, oxazole

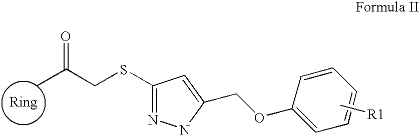

R1=ortho-, meta-, para-substituted halogens (F, Cl, Br), Me, OMe, OH, mono- and multi-substitution using combinations of aforementioned functional groups
Ring=five- or six-membered aryl rings, substituted or unsubstituted, including heterocycles; examples are benzene, pyridine, pyrimidine, pyrizine, pyrrole, pyrazole, imidazole, furan, oxazole, oxadiazole

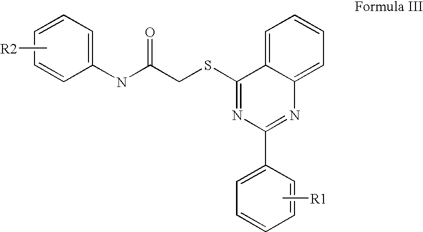

R1=ortho, meta, or para-substituted halogens (F, Cl, Br), Me, OMe, OH mono or multi substitution using combinations of aforementioned functional groups
R2=ortho, meta, or para-substituted halogens (F, Cl, Br), Me, OMe, OH, phenyl, small aromatic heterocycles including pyrrole, pyrazole, imidazole, furan, oxazole, oxadiazole and/or

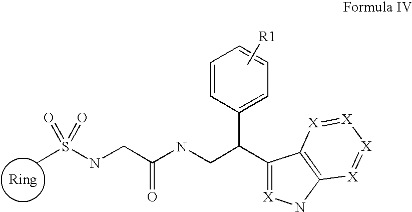

R₁=otho-, meta-, para-substituted halogens (F, CI, Br), Me, OMe, OH, mono- and multi-substitution using combinations of aforementioned functional groups
X=possible combinations of C or N
Ring=unsubstituted and substituted five- and six-membered rings including benzene, pyridine, pyrimidine, pyrizine, pyrrole, pyrazole, imidazole, furan, oxazole, oxadiazole

On the fourth aspect, the present invention provides a pharmaceutical composition comprising at least one of the compounds as stated above and a pharmaceutical carrier.

On the fifth aspect, the present invention provides a use of the inhibitor of as stated above in the treatment of cancer. Preferably, the cancer is brain tumor, lung cancer, ovarian cancer, bladder cancer, cervical cancer, colon cancer, breast cancer, and prostate cancer.

On the sixth aspect, the present invention provides a method of treating cancer in a subject comprising: administering a therapeutically effective amount of at least one of the compounds as stated above to a subject in need thereof.

On the seventh aspect, the present invention provides a method of using at least one of the compounds as stated above as an imaging agent for tumor diagnosis

### DESCRIPTION OF THE FIGURES

FIG. 1 illustrates the structure comparison of RING finger E3 and Hect E3's E2 binding domains to E2, with similarities and differences.

FIG. 2 illustrates the zoomed in view of E2 (specific residues contacting E3) and E3's E2 binding pocket.

FIG. 3 illustrates the ¹H NMR data of synthesized compound Mol11.

FIG. 4 illustrates the ¹H NMR data of synthesized compound Mol21.

FIG. 5 illustrates the 1H NMR data of synthesized compound Mol44.

FIG. 6 illustrates the 1H NMR data of synthesized compound Mol640.

FIG. 7 describes the potential derivatives of Mol 11.

FIG. 8 describes the potential derivatives of Mol 21.

FIG. 9 describes the potential derivatives of Mol 44.

FIG. 10 describes the potential derivatives of Mol 640.

FIG. 11 describes the full length sequence of E6AP from NCBI database. The region of the interest pocket is underlined.

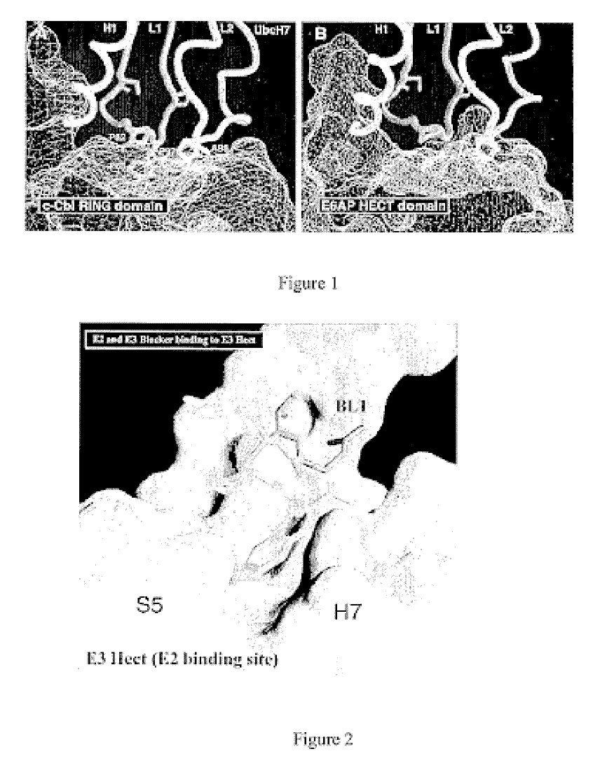

### DETAILED DESCRIPTION OF THE INVENTION

### Definition

“E2 inhibitors” in the context means “inhibitors which disrupts E2 and E3 interaction”.

### 1) Identification of Critical Binding Site for Small-Molecule Inhibitors

Protein-protein interactions are universally deemed difficult to disrupt by a small molecular agent. However, through mapping of E2-E3 interaction and molecular surface analysis of the E2-E3 interface, we've identified a binding site on E3 that appear critical for E2-E3 complex formation and suitable for a small molecule interaction. This pocket resides on E3, and is occupied by Phe63 of E2. The composing residues (from E3) of this binding site are: Val657, Leu658, Ser661, Leu662, Leu665, Met 676, Ile678, Ile682, Ile705, Phe713, Tyr717.

Lipophilic potential analysis reveals that this pocket is highly hydrophobic, with contributions from hydrophbic side chains of Leu658, Leu662, Leu665, Met 676, Ile678, Ile682, Ile705, Phe713, Tyr717 of E3.

By targeting this small hydrophobic binding pocket we will demonstrate that sufficient potency to disrupt the E2-E3 signaling cascade and consequently the ubiquitination pathway could be achieved by a small molecular agent of molecular weight around five hundred, embodying all favorable properties of a pharmaceutical agent including potency, stability (both in vitro and in vivo), efficacy and safety.

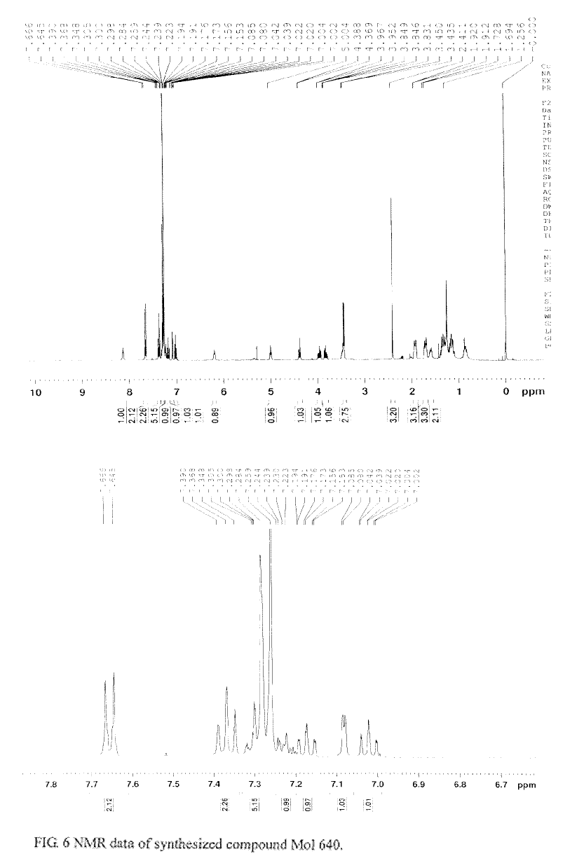

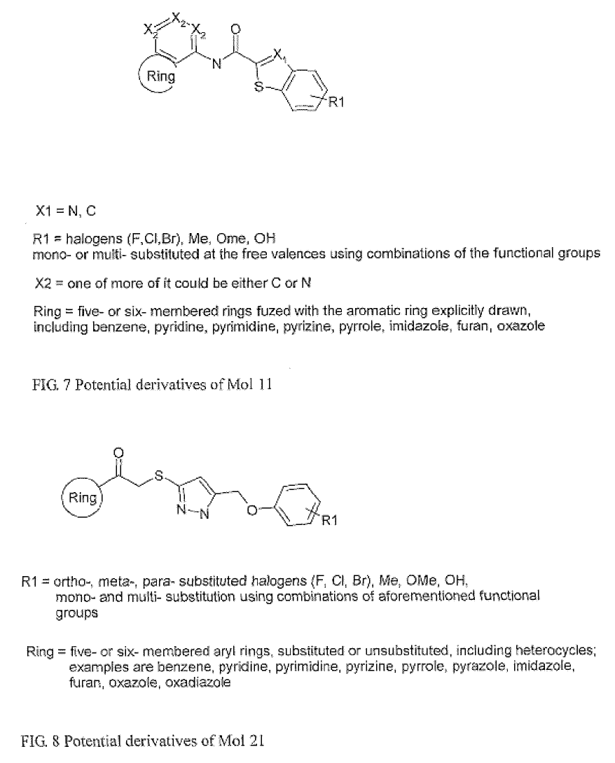

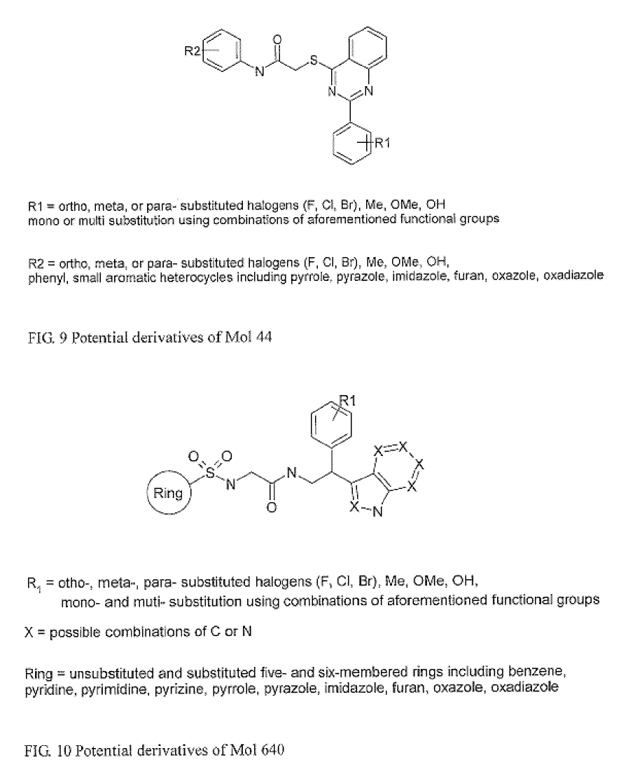

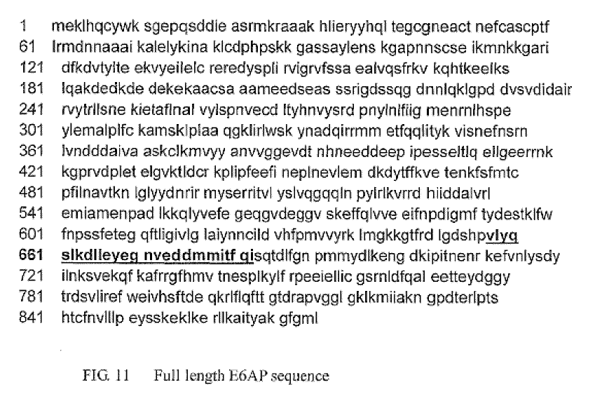

While potency could be primarily achieved by occupying the small hydrophobic pocket, we also disclose that selectivity against unwanted targets could be achieved by extending the interaction at the E2-E3 interface into the secondary binding pocket illustrated in FIG. 6. The secondary binding pocket situates adjacent to the primary hydrophobic pocket, and is comparatively less hydrophobic. It is most suited for binding of polar chemical moieties and/or combinations of heterocycles. Hydrogen bond interactions could be engaged with specific residues in the secondary pocket to further enhance potency and selectivity. It is demonstrated that by designing the optimal linkers between the two binding pockets the complementary occupation of both binding pockets could be achieved by a single chemical entity. Due to the extensive optimization toward the E2-E3 interface, the designed chemical entities are expected to highly selective against other biological targets that it may be exposed to during lifetime of action, therefore minimizing the potential side effects.

### 2) Structure-Based Drug Design

A suite of computational technologies was engaged in characterizing the interactions between E2 and E3, including protein surface generation and comparison, hydrogen bond analysis, property mappings including hydrophobicity and electrostatic potentials and overall shape complementarities at the interface of the binary complex. A small, well-defined hydrophobic pocket was discovered at the E2-E3 interface by this study, which appeared critical for the coupling interactions of the E2-E3 complex. Therefore targeting the hydrophobic pocket has the promise of disrupting the E2-E3 complex formation, leading to the intervention of the ubiquitination pathway and the subsequent therapeutic effects aforementioned.

Design of suitable inhibitors that bind into the hydrophobic pocket entailed library design, similarity search, virtual screening and de novo design. Several virtual compounds libraries based on the validated synthetic protocols were designed using the commercially available reagents. The virtual compounds were then combined with several commercial vendor collections to generate a larger compound database. The compounds were filtered on a number of druggable properties and the ones that passed all the filtering steps formed the candidate pool, which were subsequently docked into the hydrophobic pocket on E3. A free energy cutoff of −20 kJ/mol yielded several dozens of promising binders, and their binding conformations within the protein environment were visually inspected to ensure the proper binding. This set of compounds were finally optimized inside the binding site by a set of mutational operators coupled with energy evaluations to further optimize their interactions with E3 as well as implant structural novelty into the templates.

### 3) Anti-Tumor Usage Claims.

The invention provides a method of treating cancer in a subject suffering comprising administering the subject a therapeutically effective amount of these classes of compounds or any analogs thereof disclosed herein [hereafter referred to as substance] optionally in combination with a pharmaceutically suitable carrier. The method may be applied where the cancer is a solid tumor or leukemia. In particular, the method is applicable where the cancer is brain tumor, lung cancer, breast cancer, prostate cancer, ovarian cancer, or colorectal cancer.

The subject of the invention also provides a pharmaceutical composition for treating cancer comprising the substance, as an active ingredient, optionally though typically in combination with a pharmaceutically suitable carrier. The pharmaceutical compositions of the present invention may further comprise other therapeutically active ingredients, such as existing chemotherapy agents for combination therapy.

The magnitude of the therapeutic dose of the compounds of the invention will vary with the nature and severity of the condition to be treated and with the particular compound and its route of administration. Any suitable route of administration may be employed for providing a mammal, especially a human, with an effective dosage of a substance disclosed herein. For example, oral, rectal, topical, parenteral, ocular, pulmonary, nasal, etc., routes may be employed. Dosage forms include tablets, troches, dispersions, suspensions, solutions, capsules, creams, ointments, aerosols, etc.

The compositions include compositions suitable for oral, rectal, topical (including transdermal devices, aerosols, creams, ointments, lotions and dusting powders), parenteral (including subcutaneous, intramuscular, intraarterial, and intravenous), ocular (ophthalmic), pulmonary (nasal or buccal inhalation) or nasal administration. Although the most suitable route in any given case will depend largely on the nature and severity of the condition being treated and on the nature of the active ingredient, they may be conveniently presented in unit dosage form and prepared by any of the methods well known in the art of pharmacy.

In preparing oral dosage forms any of the unusual pharmaceutical media may be used, such as water, glycols, oils, alcohols, flavoring agents, preservatives, coloring agents, and the like in the case of oral liquid preparations (e.g., suspensions, elixers and solutions); or carriers such as starches, sugars, microcrystalline cellulose, diluents, granulating agents, lubricants, binders, disintegrating agents, etc., in the case of oral solid preparations are preferred over liquid oral preparations such as powders, capsules and tablets. If desired, capsules may be coated by standard aqueous or non-aqueous techniques. In addition to the dosage forms described above, the compounds of the invention may be administered by controlled release means and devices according to the general knowledge of one skilled in the art.

Pharmaceutical compositions of the present invention suitable for oral administration may be prepared as discrete units such as capsules, cachets or tablets each containing a predetermined amount of the active ingredient in powder or granular form or as a solution or suspension in an aqueous or nonaqueous liquid or in an oil-in-water or water-in-oil emulsion. Such compositions may be prepared by any of the methods known in the art of pharmacy. In general compositions are prepared by uniformly and intimately admixing the active ingredient with liquid carriers, finely divided solid carriers, or both and then, if necessary, shaping the product into the desired form. For example, a tablet may be prepared by compression or molding, optionally with one or more accessory ingredients. Compressed tablets may be prepared by compressing in a suitable machine the active ingredient in a free-flowing form such as powder or granule optionally mixed with a binder, lubricant, inert diluent or surface active or dispersing agent. Molded tablets may be made by molding in a suitable machine, a mixture of the powdered compound moistened with an inert liquid diluent.

The present invention will be better understood from the Experimental Details as following. However, one skilled in the art will understand that the examples described below is only for illustration, and will not limit the protecting scope as defined in the claims.

### EXAMPLES

### Example 1

New chemical entity (NCE) Mol11 was identified from the aforementioned procedures. Its chemical structure is depicted below.

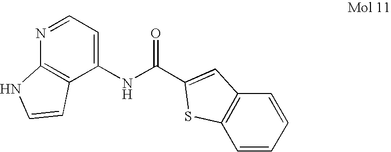

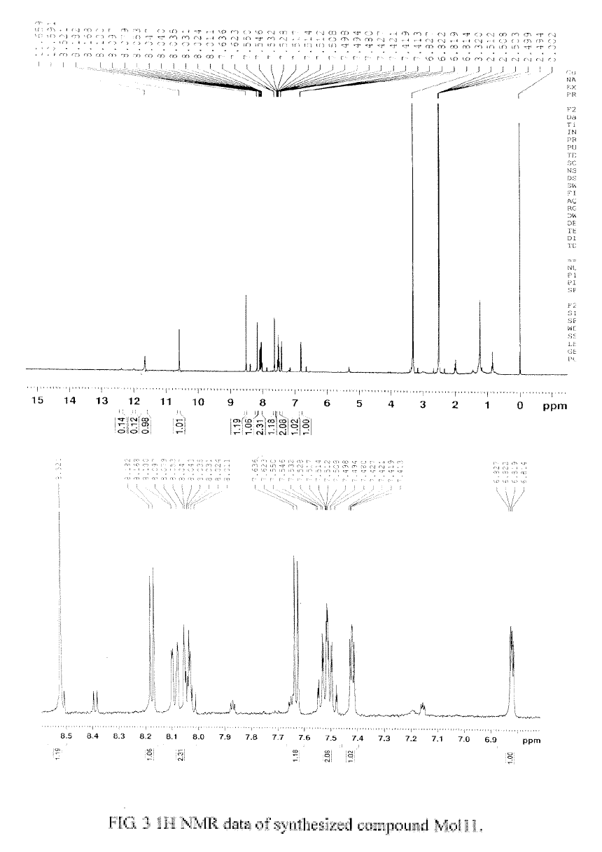

It was synthesized using the route below. Mass spectrometry data confirmed its molecular weight of 294. 1H NMR data was recorded, shown in FIG. 3.

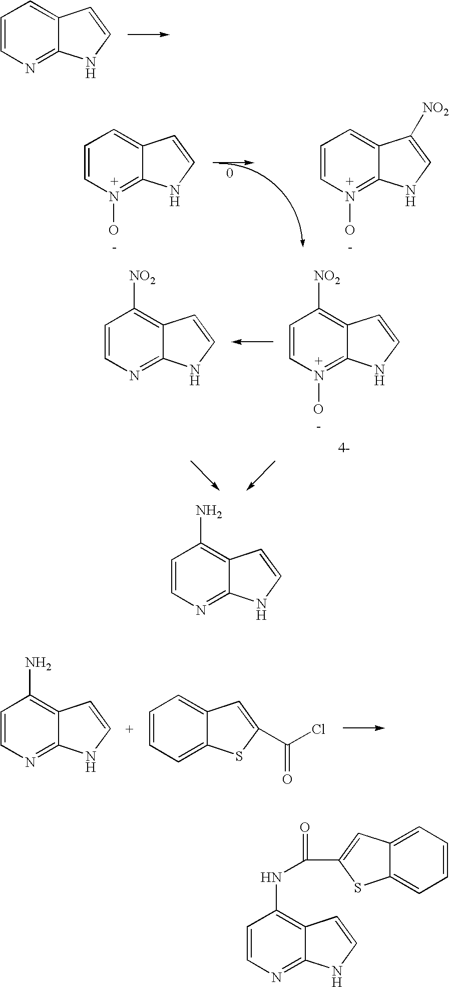

New chemical entity (NCE) Mol21 was identified from the aforementioned procedures. Its chemical structure is depicted below.

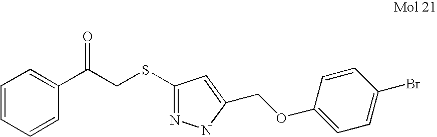

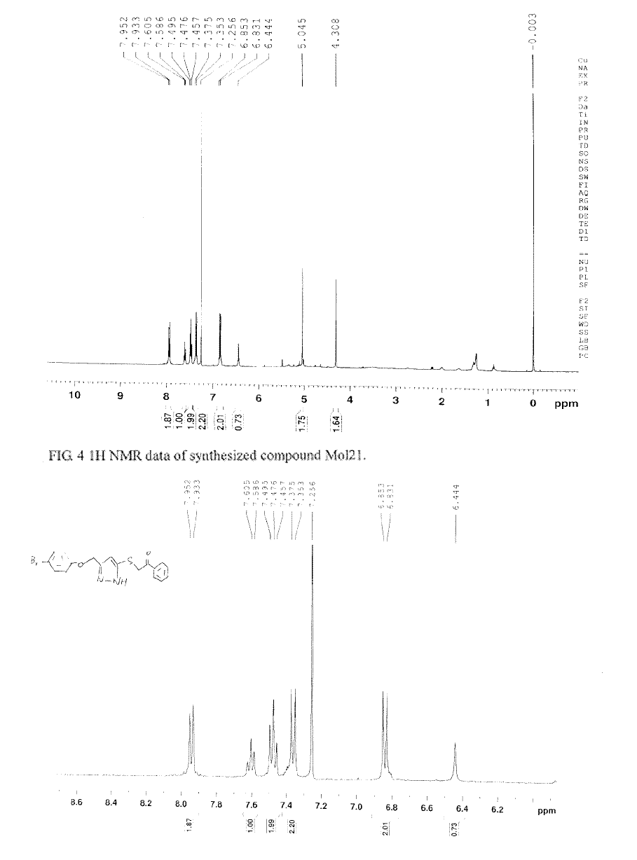

It was synthesized using the route below. Mass spectrometry data confirmed its molecular weight of 403. ¹H NMR data was recorded, shown in FIG. 4.

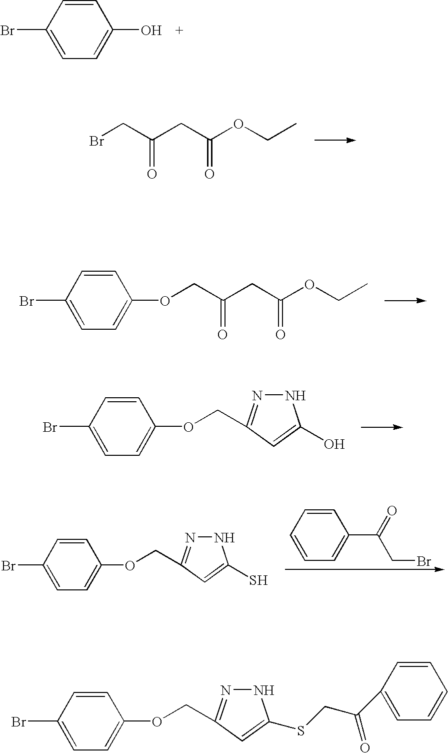

### Example 3

New chemical entity (NCE) Mol44 was identified from the aforementioned procedures. Its chemical structure is depicted below.

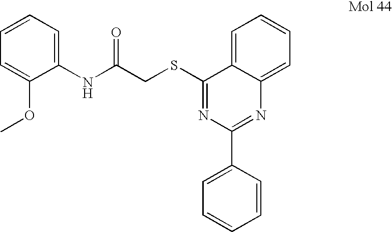

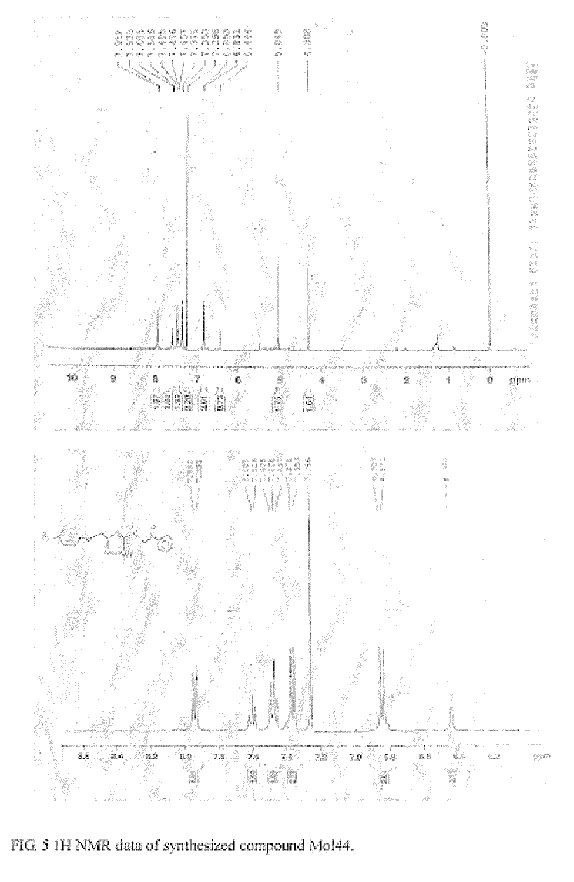

It was synthesized using the route below. Mass spectrometry data confirmed its molecular weight of 402. 1H NMR data was recorded, shown in FIG. 5.

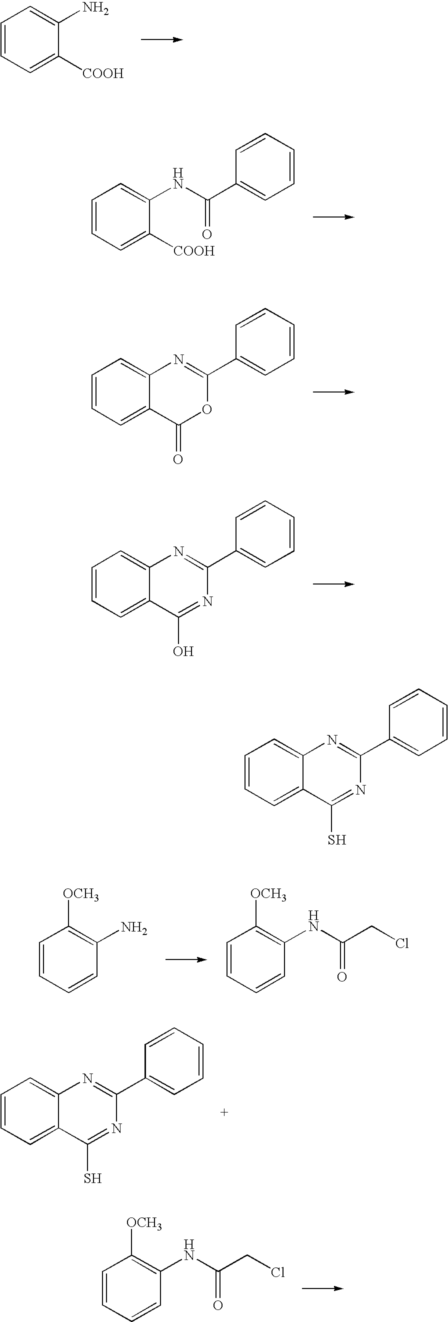

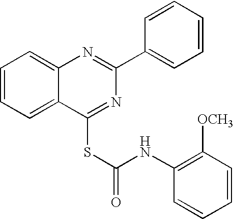

### Example 4

New chemical entity (NCE) Mol640 was identified from the aforementioned procedures. Its chemical structure is depicted below.

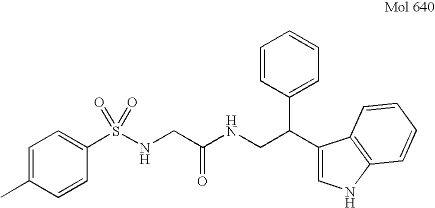

It was synthesized using the route below. Mass spectrometry data confirmed its molecular weight of 448. 1H NMR data was recorded, shown in FIG. 6.

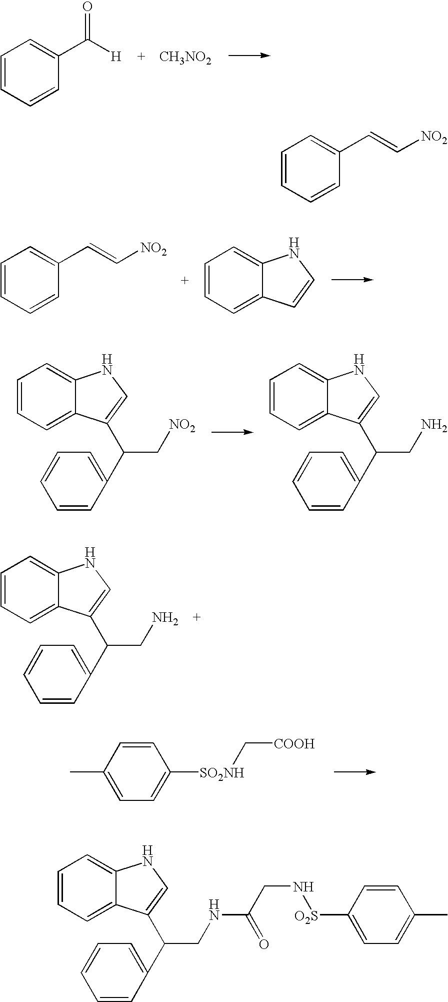

### Example 5

The effect of the above identified compounds on anti-tumor activity in human carcinoma cells was determined by the MTT survival assay. The MTT assay is a commonly used method in evaluation of cell survival, based on the ability of viable cells to convert MTT (MTT, Sigma, Cat No. 044K5307), a soluble tetrazolium salt [3-(4,5-dimethylthuazole-2-yl)-2,5 diphenyl tetrazolium bromide], into an insoluble formazan precipitate, which is quantitated by spectrophotometry following solubilization in dimethyl sulfoxide (DMSO).

In brief, different carcinoma cells were treated with negative control, various test articles at different concentration, and positive control (DDP), in 96-well tissue culture dishes. Each test article at different concentration is repeated 3 times.

Different carcinoma cells were diluted with 10% FBS and RPM1640 or MEM media to a concentration of 3-5×10⁴ per ml suspension. 100 microliter of cells were incubated in each well in 96-well tissue culture dishes at 37 C. The cells were incubated at 37 C on the first day. On the second day, media supernatant was removed and replaced with media and different concentration of test articles (100 microliter in each well). Three Negative control wells and three positive control (DDP) wells were also included. The 96-well plate were incubated at 37 C for another 68 hours. After 68 hours, supernatant of each well was removed. 100 microliter of 1 mg/ml MTT diluted in PBS was injected into each well. The cells were incubated at 37 C for another 4 hours. The cells were then solubilized in 150 microliter DMSO, mixed for 10 minutes and absorbance readings were taken using Microplate reader Bio-Rad Model 550 at 570 nm. IR (cell growth inhibition rate) % was calculated as (OD570 of negative control-OD570 with test article)/OD570 of negative control. IC50 (cell 50% inhibition concentration) at 95% confidence level was calculated with Bliss method.

### Cervical Cancer Line (Hela Cells) IC50 Results

| | | | | IC₅₀ |
|---|---|---|---|---|
| | | | | (95% |
| | Concentration | | | confidence |
| Test Article | (μg/ml) | OD(A570 nm) | IR (%) | level) |
| Negative | 0 | 0.743 ± 0.045 | | |
| Control | | | | |
| Mol 44 | 16.67 | 0.688 ± 0.045 | 7.4 | N/A |
| | 5.56 | 0.671 ± 0.044 | 9.7 | |
| | 1.85 | 0.737 ± 0.113 | 0.8 | |
| | 0.62 | 0.765 ± 0.200 | — | |
| | 0.21 | 0.655 ± 0.007 | 11.8 | |
| | 0.07 | 0.599 ± 0.109 | 19.3 | |
| Mol 11 | 100 | 0.145 ± 0.031 | 80.5 | 31.85 |
| | 33.33 | 0.283 ± 0.047 | 61.9 | 26.78-38.33 |
| | 11.11 | 0.653 ± 0.134 | 12.0 | |
| | 3.70 | 0.723 ± 0.032 | 2.6 | |
| | 1.23 | 0.790 ± 0.037 | — | |
| | 0.41 | 0.738 ± 0.080 | 0.7 | |
| Mol 21 | 100 | 0.130 ± 0.039 | 82.5 | 22.23 |
| | 33.33 | 0.194 ± 0.031 | 73.8 | 18.80-26.44 |
| | 11.11 | 0.480 ± 0.035 | 35.4 | |
| | 3.70 | 0.774 ± 0.066 | — | |
| | 1.23 | 0.785 ± 0.081 | — | |
| | 0.41 | 0.668 ± 0.049 | 10.0 | |
| Mol 640 | 100 | 0.078 ± 0.023 | 89.5 | 25.26 |
| | 33.33 | 0.172 ± 0.010 | 76.8 | 21.94-29.12 |
| | 11.11 | 0.652 ± 0.057 | 12.2 | |
| | 3.70 | 0.819 ± 0.085 | — | |
| | 1.23 | 0.826 ± 0.101 | — | |
| | 0.41 | 0.651 ± 0.044 | 12.3 | |
| DDP (cisplatin) | 4 | 0.078 ± 0.001 | 89.5 | 0.105 |
| (positive/ | 1 | 0.123 ± 0.007 | 83.4 | 0.062-0.156 |
| control) | 0.25 | 0.206 ± 0.014 | 72.2 | |
| | 0.0625 | 0.481 ± 0.081 | 35.3 | |

### Breast Cancer Cell Line (MCF-7) IC50 Results

| | | | | IC₅₀ |
|---|---|---|---|---|
| | | | | (95% |
| | Concentration | | | confidence |
| Test Article | (μg/ml) | OD(A570 nm) | IR (%) | level) |
| Negative | 0 | 0.903 ± 0.083 | | |
| Control | | | | |
| Mol 44 | 50 | 0.527 ± 0.035 | 41.6 | 145.21 |
| | 25 | 0.587 ± 0.030 | 34.9 | 71.33-551.08 |
| | 12.5 | 0.791 ± 0.151 | 12.4 | |
| | 6.25 | 0.791 ± 0.151 | 10.2 | |
| | 3.125 | 0.811 ± 0.109 | 12.8 | |
| | 1.56 | 0.787 ± 0.176 | 13.3 | |
| Mol 11 | 100 | 0.478 ± 0.079 | 47.0 | 94.23 |
| | 50 | 0.490 ± 0.093 | 45.7 | 66.90-154.16 |
| | 25 | 0.705 ± 0.076 | 21.9 | |
| | 12.5 | 0.752 ± 0.164 | 16.6 | |
| | 6.25 | 0.711 ± 0.045 | 21.2 | |
| | 3.125 | 0.903 ± 0.194 | 0 | |
| Mol 21 | 100 | 0.072 ± 0.007 | 92.1 | 9.93 |
| | 50 | 0.069 ± 0.006 | 92.4 | 8.53-11.45 |
| | 25 | 0.098 ± 0.006 | 89.1 | |
| | 12.5 | 0.346 ± 0.022 | 61.6 | |
| | 6.25 | 0.624 ± 0.008 | 30.9 | |
| | 3.125 | 0.789 ± 0.064 | 12.5 | |
| Mol 640 | 100 | 0.069 ± 0.007 | 92.3 | 14.12 |
| | 50 | 0.098 ± 0.012 | 89.1 | 12.34-16.11 |
| | 25 | 0.094 ± 0.012 | 89.6 | |
| | 12.5 | 0.649 ± 0.036 | 28.1 | |
| | 6.25 | 0.708 ± 0.058 | 21.5 | |
| | 3.125 | 0.807 ± 0.016 | 10.5 | |
| DDP (cisplatin) | 4 | 0.107 ± 0.007 | 88.2 | 0.596 |
| (positive/ | 1 | 0.151 ± 0.011 | 83.3 | 0.483-0.737 |
| control) | 0.25 | 0.879 ± 0.059 | 2.6 | |
| | 0.0625 | 0.787 ± 0.005 | 12.8 | |

### Colon Cancer Cell Line (HCT116) IC50 Results

| | | | | IC₅₀ |
|---|---|---|---|---|
| | | | | (95% |
| | Concentration | | | confidence |
| Test Article | (μg/ml) | OD(A570 nm) | IR (%) | level) |
| Negative | 0 | 0.542 ± 0.027 | | |
| Control | | | | |
| Mol 21 | 100 | 0.083 ± 0.001 | 84.7 | 3.160 |
| | 50 | 0.085 ± 0.004 | 84.3 | 2.992-3.330 |
| | 25 | 0.101 ± 0.020 | 81.3 | |
| | 12.5 | 0.150 ± 0.025 | 72.3 | |
| | 6.25 | 0.300 ± 0.050 | 44.6 | |
| | 3.125 | 0.237 ± 0.098 | 56.2 | |
| | 100 | 0.086 ± 0.012 | 84.1 | |
| | 50 | 0.083 ± 0.011 | 84.6 | |
| | 25 | 0.109 ± 0.006 | 80.0 | |
| | 12.5 | 0.267 ± 0.035 | 50.7 | |
| | 6.25 | 0.394 ± 0.039 | 27.3 | |
| | 3.125 | 0.377 ± 0.025 | 30.4 | |
| DDP | 4 | 0.133 ± 0.016 | 75.5 | 0.234 |
| | 1 | 0.201 ± 0.060 | 62.9 | 0.211-0.257 |
| | 0.25 | 0.266 ± 0.016 | 51.0 | |

### Lung Cancer Cell Line (A459) IC50 Results

| | | | | IC₅₀ |
|---|---|---|---|---|
| | | | | (95% |
| | Concentration | | | confidence |
| Test Article | (μg/ml) | OD(A570 nm) | IR (%) | level) |
| Negative | 0 | 0.637 ± 0.044 | | |
| Control | | | | |
| Mol 44 | 50 | 0.368 ± 0.039 | 42.3 | 150 |
| | 25 | 0.504 ± 0.046 | 21.0 | |
| | 12.5 | 0.586 ± 0.051 | 8.1 | |
| | 6.25 | 0.680 ± 0.015 | −6.8 | |
| | 3.125 | 0.786 ± 0.048 | −23.3 | |
| | 1.56 | 0.634 ± 0.009 | 0.6 | |
| Mol 11 | 100 | 0.108 ± 0.013 | 83.1 | 19.435 |
| | 50 | 0.152 ± 0.017 | 76.1 | 19.116-19.759 |
| | 25 | 0.230 ± 0.040 | 64.0 | |
| | 12.5 | 0.438 ± 0.051 | 31.2 | |
| | 6.25 | 0.471 ± 0.001 | 26.1 | |
| | 3.125 | 0.583 ± 0.015 | 8.6 | |
| DDP | 4 | 0..185 ± 0.231 | 71.0 | 1.162 |
| | 1 | 0.341 ± 0.060 | 46.4 | 1.113-1.213 |
| | 0.25 | 0.517 ± 0.073 | 18.8 | |
| | 0.0625 | 0.485 ± 0.046 | 23.9 | |

### Lung Cancer A459 Cell Line Experiment #2

| | | | | IC₅₀ |
|---|---|---|---|---|
| | | | | (95% |
| | Concentration | | | confidence |
| Test Article | (μg/ml) | OD(A570 nm) | IR (%) | level) |
| Negative | 0 | 0.637 ± 0.044 | | |
| Control | | | | |
| Mol 21 | 100 | 0.082 ± 0.015 | 87.1 | 6.720 |
| | 50 | 0.069 ± 0.010 | 89.2 | 6.303-7.134 |
| | 25 | 0.118 ± 0.027 | 81.5 | |
| | 12.5 | 0.279 ± 0.014 | 56.2 | |
| | 6.25 | 0.662 ± 0.031 | −3.9 | |
| | 3.125 | 0.642 ± 0.073 | −0.7 | |
| Mol 640 | 100 | 0.082 ± 0.014 | 87.1 | 25.828 |
| | 50 | 0.081 ± 0.010 | 87.2 | 25.516-26.141 |
| | 25 | 0.232 ± 0.006 | 63.6 | |
| | 12.5 | 0.593 ± 0.034 | 6.9 | |
| | 6.25 | 0.717 ± 0.020 | −12.5 | |
| | 3.125 | 0.781 ± 0.040 | −22.6 | |
| DDP | 4 | 0..185 ± 0.231 | 71.0 | 1.162 |
| | 1 | 0.341 ± 0.060 | 46.4 | 1.114-1.213 |
| | 0.25 | 0.517 ± 0.073 | 18.8 | |
| | 0.0625 | 0.485 ± 0.046 | 23.9 | |

### Liver Cancer Cell Line (SMMC-7721) IC50 Results

| | | | | IC₅₀ |
|---|---|---|---|---|
| | | | | (95% |
| | Concentration | | | confidence |
| Test Article | (μg/ml) | OD(A570 nm) | IR (%) | level) |
| Negative | 0 | 0.440 ± 0.049 | | |
| Control | | | | |
| Mol 44 | 50 | 0.345 ± 0.025 | 21.6 | |
| | 25 | 0.373 ± 0.019 | 15.4 | |
| | 12.5 | 0.392 ± 0.039 | 11.1 | |
| | 6.25 | 0.272 ± 0.019 | 38.3 | |
| | 3.125 | 0.321 ± 0.088 | 27.2 | |
| | 1.56 | 0.275 ± 0.004 | 37.6 | |
| Mol 11 | 100 | 0.141 ± 0.006 | 68.1 | 14.934 |
| | 50 | 0.163 ± 0.009 | 63.0 | 14.338-15.549 |
| | 25 | 0.205 ± 0.018 | 53.5 | |
| | 12.5 | 0.207 ± 0.007 | 53.1 | |
| | 6.25 | 0.286 ± 0.092 | 35.1 | |
| | 3.125 | 0.277 ± 0.018 | 37.0 | |
| DDP | 4 | 0.169 ± 0.022 | 61.6 | |
| | 1 | 0.128 ± 0.011 | 71.0 | |
| | 0.25 | 0.237 ± 0.020 | 46.2 | |
| | 0.0625 | 0.570 ± 0.166 | −29.4 | |

### Liver Cancer SMMC-7721 Cell Line Experiment #2

| | | | | IC₅₀ |
|---|---|---|---|---|
| | | | | (95% |
| | Concentration | | | confidence |
| Test Article | (μg/ml) | OD(A570 nm) | IR (%) | level) |
| Negative | 0 | 0.461 ± 0.271 | | |
| Control | | | | |
| Mol 21 | 100 | 0.077 ± 0.009 | 83.2 | 9.515 |
| | 50 | 0.079 ± 0.005 | 82.9 | 9.321-9.712 |
| | 25 | 0.129 ± 0.006 | 72.0 | |
| | 12.5 | 0.146 ± 0.041 | 68.3 | |
| | 6.25 | 0.161 ± 0.085 | 65.1 | |
| | 3.125 | 0.690 ± 0.603 | −49.9 | |
| Mol 640 | 100 | 0.098 ± 0.014 | 78.7 | 8.018 |
| | 50 | 0.091 ± 0.002 | 80.2 | 7.681-8.359 |
| | 25 | 0.200 ± 0.002 | 56.6 | |
| | 12.5 | 0.268 ± 0.013 | 41.9 | |
| | 6.25 | 0.278 ± 0.013 | 39.8 | |
| | 3.125 | 0.215 ± 0.008 | 53.4 | |
| DDP | 4 | 0.244 ± 0.155 | 47.0 | 4.155 |
| | 1 | 0.262 ± 0.020 | 43.1 | 3.757-4.640 |
| | 0.25 | 0.348 ± 0.045 | 24.5 | |

### Ovarian Cancer Cell Line (SKOV3) IC50 Results

| | | | | IC₅₀ |
|---|---|---|---|---|
| | | | | (95% |
| | Concentration | | | confidence |
| Test Article | (μg/ml) | OD(A570 nm) | IR (%) | level) |
| Negative | 0 | 0.701 ± 0.035 | | |
| control | | | | |
| Mol44 | 50 | 0.333 ± 0.008 | 52.4 | 60.00 |
| | 25 | 0.426 ± 0.008 | 39.1 | |
| | 12.5 | 0.525 ± 0.035 | 25.1 | |
| | 6.25 | 0.539 ± 0.023 | 23.0 | |
| | 3.125 | 0.533 ± 0.017 | 23.9 | |
| | 1.56 | 0.505 ± 0.029 | 27.9 | |
| Mol11 | 100 | 0.098 ± 0.010 | 86.0 | 29.00 |
| | 50 | 0.314 ± 0.041 | 55.1 | |
| | 25 | 0.385 ± 0.017 | 45.0 | |
| | 12.5 | 0.671 ± 0.063 | 4.1 | |
| | 6.25 | 0.694 ± 0.043 | 0.9 | |
| | 3.125 | 0.753 ± 0.074 | 0 | |
| DDP | 4 | 0.083 ± 0.002 | 88.2 | 0.89 |
| | 1 | 0.267 ± 0.044 | 62.0 | |
| | 0.25 | 0.643 ± 0.141 | 8.3 | |
| | 0.0625 | 0.689 ± 0.065 | 1.6 | |

### Ovarian Cancer Cell Line (SKOV3) IC50 Results

| | | | | IC₅₀ |
|---|---|---|---|---|
| | | | | (95% |
| | Concentration | | | confidence |
| Test Article | (μg/ml) | OD(A570 nm) | IR (%) | level) |
| Negative | 0 | 0.701 ± 0.035 | | |
| control | | | | |
| Mol21 | 100 | 0.077 ± 0.011 | 89.1 | 17.69 |
| | 50 | 0.119 ± 0.010 | 83.0 | |
| | 25 | 0.429 ± 0.002 | 38.8 | |
| | 12.5 | 0.460 ± 0.195 | 34.3 | |
| | 6.25 | 0.532 ± 0.025 | 24.1 | |
| | 3.125 | 0.503 ± 0.031 | 28.2 | |
| Mol640 | 100 | 0.079 ± 0.010 | 88.7 | 26.87 |
| | 50 | 0.082 ± 0.008 | 88.3 | |
| | 25 | 0.394 ± 0.037 | 43.8 | |
| | 12.5 | 0.632 ± 0.020 | 9.7 | |
| | 6.25 | 0.628 ± 0.025 | 10.3 | |
| | 3.125 | 0.716 ± 0.048 | 0 | |
| DDP | 4 | 0.083 ± 0.002 | 88.2 | 0.89 |
| | 1 | 0.267 ± 0.044 | 62.0 | |
| | 0.25 | 0.643 ± 0.141 | 8.3 | |
| | 0.0625 | 0.689 ± 0.065 | 1.6 | |

### Glioma Cell Line (Brain Tumor U251) IC50 Results

| | | | | IC₅₀ |
|---|---|---|---|---|
| | | | | (95% |
| | Concentration | | | confidence |
| Test Article | (μg/ml) | OD(A570 nm) | IR (%) | level) |
| Negative | 0 | 0.877 ± 0.055 | | |
| control | | | | |
| Mol44 | 50 | 0.659 ± 0.047 | 24.9 | 129.14 |
| | 25 | 0.737 ± 0.067 | 15.9 | |
| | 12.5 | 0.811 ± 0.050 | 7.5 | |
| | 6.25 | 0.866 ± 0.090 | 1.2 | |
| | 3.125 | 0.705 ± 0.061 | 19.6 | |
| | 1.56 | 0.790 ± 0.086 | 9.9 | |
| Mol11 | 100 | 0.650 ± 0.006 | 25.8 | 4340 |
| | 50 | 0.709 ± 0.054 | 19.1 | |
| | 25 | 0.697 ± 0.053 | 20.5 | |
| | 12.5 | 0.744 ± 0.045 | 15.1 | |
| | 6.25 | 0.758 ± 0.019 | 13.5 | |
| | 3.125 | 0.797 ± 0.016 | 9.1 | |
| DDP | 4 | 0.174 ± 0.009 | 80.2 | 1.13 |
| | 1 | 0.365 ± 0.058 | 58.4 | |
| | 0.25 | 0.688 ± 0.035 | 21.5 | |
| | 0.0625 | 0.729 ± 0.090 | 16.8 | |

### Glioma Cell Line (Brain Tumor U251) IC50 Results

| | | | | IC₅₀ |
|---|---|---|---|---|
| | | | | (95% |
| | Concentration | | | confidence |
| Test Article | (μg/ml) | OD(A570 nm) | IR (%) | level) |
| Negative | 0 | 0.877 ± 0.055 | | |
| control | | | | |
| Mol21 | 100 | 0.094 ± 0.007 | 89.2 | 16.24 |
| | 50 | 0.121 ± 0.019 | 86.2 | |
| | 25 | 0.232 ± 0.034 | 73.5 | |
| | 12.5 | 0.501 ± 0.058 | 42.8 | |
| | 6.25 | 0.719 ± 0.110 | 18.0 | |
| | 3.125 | 0.840 ± 0.013 | 4.2 | |
| Mol640 | 100 | 0.090 ± 0.014 | 89.7 | 11.65 |
| | 50 | 0.087 ± 0.012 | 90.1 | |
| | 25 | 0.085 ± 0.012 | 90.3 | |
| | 12.5 | 0.354 ± 0.021 | 59.6 | |
| | 6.25 | 0.696 ± 0.054 | 20.6 | |
| | 3.125 | 0.796 ± 0.055 | 9.2 | |
| DDP | 4 | 0.174 ± 0.009 | 80.2 | 1.13 |
| | 1 | 0.365 ± 0.058 | 58.4 | |
| | 0.25 | 0.688 ± 0.035 | 21.5 | |
| | 0.0625 | 0.729 ± 0.090 | 16.8 | |

All patents, patent applications, and literature references referred to herein are hereby incorporated by reference in their entirety. Many variations of the present invention will suggest themselves to those skilled in the art in light of the above detailed description. Such obvious variations are within the full-intended scope of the appended claims.

Note: E3 binding pocket for design is surrounded by the following residues Val634, Leu635, Ser638, Leu639, Leu642, Met 653, Ile655, Ile659, Ile682, Phe690, Tyr694, in E6AP 3-D structure in Huang et. al. In full length E6AP (or UBE3A_human), these residues are Val657, Leu658, Ser661, Leu662, Leu665, Met 676, Ile678, Ile682, Ile705, Phe713, Tyr717.

### REFERENCES CITED

Balasubramanyam M. et al. Mol. Cell. Biochem. 275(1-2), 117-25 (2005)
Bueso-Ramos, C. E. et al. Blood 82, 2617-2623 (1993).
Fuchs, S. Y., Adler, V., Buschmann, T., Wu, X. & Ronai, Z. Oncogene 17, 2543-2547 (1998).
Gustafsson, B. & Stal, O. Pediatr Hematol Oncol 15, 519-526 (1998).
Hall, E. J. Int J Radiat Oncol Biol Phys 30, 1019-1028 (1994).
Hochstrasser, M. Annu Rev Genet. 30, 405-439 (1996).
Honda, R., Tanaka, H. & Yasuda, H. FEBS Lett 420, 25-27 (1997).
Huang, L. et al. Science 286, 1321-1326 (1999).
Keating, M. J. Clin Cancer Res 3, 2598-2604 (1997).
Konstantinopoulos P A et al. Expert Opin Investig Drugs 15(9), 1067-75 (2006)
Magrath, I. Cancer Res 52, 5529s-5540s (1992).
Masdehors, P. et al. Blood 96, 269-274 (2000).
Montagut C. et al. Clin Transl Oncol. 8(5): 313-7 (2006)
Oren, M. Semin Cancer Biol 5, 221-227 (1994).
Pan, Y. & Haines, D. S. Cancer Res 59, 2064-2067 (1999).
Pulczynski, S. Leuk Lymphoma 15, 243-252 (1994).
Scheffner, M., Smith, S. & Jentsch, S. in Ubiquitin and the Biology of the Cell, Plenum, N.Y., Peters, J.-M., Harris, J. R., and Finley, D., 65-98 (1998).
Schwarz, S. E., Rosa, J. L. & Scheffner, M. J Biol Chem 273, 12148-12154 (1998).
Soussi, T. & Jonveaux, P. Nouv Rev Fr Hematol 33, 477-480 (1991).
Starita L M. et al. Cancer Biol Ther 5(2), 137-141 (2006)
Teoh, G. et al. Blood 90, 1982-1992 (1997).
Voutsadakis, I. A. Acta Oncol 39, 151-156 (2000).
Williams A J et al. Neurochem Int. 49(2), 106-12 (2006)
Zheng, N. et al. Cell 102, 533-539 (2000)

1. A protein binding pocket for the design of E2 inhibitors comprising Val657, Leu658, Ser661, Leu662, Leu665, Met 676, Ile678, Ile682, Ile705, Phe713, and Tyr717 of SEQ ID NO. 1 or a virtual representation thereof.

2. A process for identifying E2 inhibitors, wherein the process comprises:
providing a virtual representation of the protein binding pocket of claim 1;
providing a plurality of virtual compounds;
docking said plurality of virtual compounds with said protein binding pocket; and
selecting virtual compounds that bind in said protein binding pocket.

3. A E2 inhibitor, wherein said inhibitor comprises a compound selected from group consisting of: 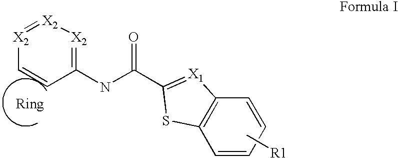
X1=N,C
R1=halogens (F,Cl,Br), Me, Ome, OH
mono- or multi-substituted at the free valences using combinations of the functional groups
X2=one of more of it could be either C or N
Ring=five- or six-membered rings fuzed with the aromatic ring explicitly drawn, including benzene, pyridine, pyrimidine, pyrizine, pyrrole, imidazole, furan, oxazole 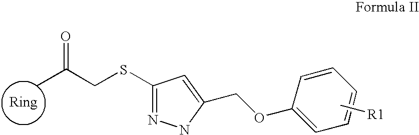
R1=ortho-, meta-, para-substituted halogens (F, Cl, Br), Me, OMe, OH,
mono- and multi-substitution using combinations of aforementioned functional groups
Ring=five- or six-membered aryl rings, substituted or unsubstituted, including heterocycles; examples are benzene, pyridine, pyrimidine, pyrizine, pyrrole, pyrazole, imidazole, furan, oxazole, oxadiazolo; 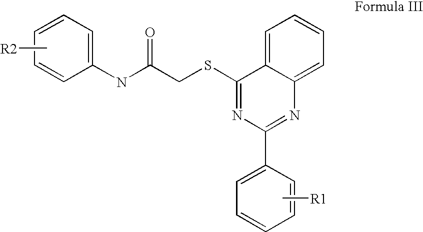
R1=ortho, meta, or para-substituted halogens (F, Cl, Br), Me, OMe, OH mono or multi substitution using combinations of aforementioned functional groups
R2=ortho, meta, or para-substituted halogens (F, Cl, Br), Me, OMe, OH, phenyl, small aromatic heterocycles including pyrrole, pyrazole, imidazole, furan, oxazole, oxadiazole; and 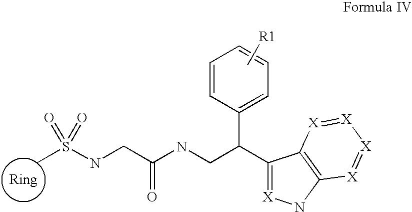
R₁=otho-, meta-, para-substituted halogens (F, Cl, Br), Me, OMe, OH, mono- and multi-substitution using combinations of aforementioned functional groups
X=possible combinations of C or N
Ring=unsubstituted and substituted five- and six-membered rings including benzene, pyridine, pyrimidine, pyrizine, pyrrole, pyrazole, imidazole, furan, oxazole, oxadiazole.

4. A pharmaceutical composition comprising at least one of the compounds of claim 3 and a pharmaceutical carrier.

5. A method of reducing cancer cell survival comprising:
providing at least one of the compounds of claim 3;
contacting a cancer cell with said compound; and
evaluating the survival of said cancer cell.

6. The method of claim 5, wherein the cancer cell is selected from the group consisting of a brain tumor cell, a lung cancer cell, an ovarian cancer cell, a bladder cancer cell, a cervical cancer cell, a colon cancer cell, a breast cancer cell, and a prostate cancer cell.

7. A method of treating cancer in a subject comprising:
administering a therapeutically effective amount of at least one of the compounds of claim 3 to a subject in need thereof.

8. The method of claim 7, further comprising the step of administering a therapeutically effective amount of a chemotherapy agent within the therapeutic window for this chemotherapy agent to said patient to achieve a therapeutically effective change in progression of a cancer selected from the group consisting of brain tumor, lung cancer, ovarian cancer, bladder cancer, cervical cancer, colon cancer, breast cancer, and prostate cancer.

9. The method of claim 7, further comprising the step of administering a therapeutically dose of radiotherapy within the therapeutic window for this radiotherapy to said patient to achieve a therapeutically effective change in progression of a cancer selected from the group consisting of brain tumor, lung cancer, ovarian cancer, bladder cancer, cervical cancer, colon cancer, breast cancer, and prostate cancer.

10. The method of claim 7 wherein said method further comprises providing a chemotherapeutic agent to said subject.

11. A method of tumor diagnosis comprising:
providing one of the compounds of claim 3 to a subject; and
performing imaging to detect said compound and make a tumor diagnosis.


SEQUENCE LISTING (1 sequence)

SEQ ID NO: 1 - protein, 875 aa, Human
MEKLHQCYWKSGEPQSDDIEASRMKRAAAKHLIERYYHQLTEGCGNEACTNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNLQKLGPDDVSVDIDAIRRVYTRLLSNEKIETAFLNALVYLSPNVECDLTYHNVYSRDPNYLNLFIIGMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGML